(19) United States
(12) Patent Application Publication
(10) Pub. No.: US 2024/0382085 A1
(43) Pub. Date: Nov. 21, 2024

(54) MONITORING AND ACTIVITY MATCHING SYSTEM FOR EYE HEALTH

(71) Applicant: Chih-Kai LIANG, Taoyuan City (TW)

(72) Inventor: Chih-Kai LIANG, Taoyuan City (TW)

(21) Appl. No.: 18/664,371

(22) Filed: May 15, 2024

(30) Foreign Application Priority Data
May 15, 2023 (TW) 112117981

(51) Int. Cl. A61B 3/10; G16H 20/00

(52) U.S. Cl. CPC A61B 3/1005; G16H 20/00

(57) ABSTRACT

The present invention provides a the monitoring and activity matching system for eye health includes an eye axial length detecting module used for detecting the data of an eye axial length of a patient, a member system used for inputting the data of the eye axial length, and a follow-up consulting and activity matching system used for allowing the patient to be matched with a follow-up consulting and an activity according to the data of the eye axial length.

### CROSS REFERENCE TO RELATED APPLICATION

The present application claims priority to Taiwan patent application Serial No. 112117981 filed on May 15, 2023 the entire content of which is incorporated by reference to this application.

### BACKGROUND OF THE INVENTION

### 1. Field of the Invention

The present invention relates to a monitoring and activity matching system, epically to a monitoring and activity matching system for eye health.

### 2. Description of the Prior Art

The ability to detect and monitor myopia at an early stage through conventional

technology encounters notable challenges. Patients, especially young ones, are required to visit ophthalmologists or optometrists at specialized hospitals or clinics for frequent examinations, which becomes problematic due to the limited number of such facilities. In Taiwan, for example, myopia affects between 20 to 25% of first graders, with this figure rising dramatically to 80 to 85% by the twelfth grade, overwhelming the available specialized eye care facilities. Similarly, in the United

States, although the incidence of myopia among school children is not as high, the increasing use of electronic devices has led to a rise in myopia rates, creating a situation comparable to that in Taiwan. Additionally, diagnosing early-stage myopia in young patients is further complicated as it requires the administration of mydriatic eyedrops to fully relax any potential excess accommodation for precise measurements. This procedure can take about an hour and may cause discomfort, thus deterring regular follow-up.

Moreover, focusing on Taiwan, although there are about 5,000 optometry institutions and glasses shops spread across the country, these primarily concentrate on retail, such as selling eyeglass frames, rather than providing eye health services. This focus on sales leads to off-peak periods with unnecessary operational expenses, and the professional optometry staff may not always be equipped to deliver adequate eye health services to evaluate the patient's current state or future health care needs. The situation is even more challenging in the U.S., where long distances and difficult scheduling further complicate long-term examination and tracking. Despite the willingness of optometry institutions and glasses shops to collaborate with ophthalmologists or optometrists, there is currently no systematic solution to effectively bridge this gap.

The first prior art has disclosed a system for replicating a standardized visual acuity test (such as the Snellen test) comprising a binocular viewer attached to a smartphone. The binocular viewer comprises a housing comprising a pair tube covers having voids allowing to view through a pair of lens tubes with each lens tube in visual communication with a second lens, a first lens, an aperture and a front cover. The optical systems use an artful combination of front and back lens surfaces, demagnification and other systems to faithfully replicate the sight lines perceived by a user of a traditional test. The system also allows the incorporation of other tests conducted with both eyes including color sensitivity and contrast; furthermore, by placing a deformable, tunable lens between the second lens and the eye, the device serves as an ophthalmic refractometer, allowing a spherical equivalent refraction estimate for each eye.

The first prior art also discloses a publicity unit which has features of non-obviousness and novelty that overcome the disadvantages existed in the conventional technology. The unit includes two lens sets having the optical characteristic which is suitable for optically generating a normal visual acuity test within a relative short distance of the binocular viewer. The term “visual acuity” can be defined as a capability of the eyes seeing the details within a preset distance. The embodiment of this cited prior art utilizes a high resolution screen of the smartphone to provide a light source capable being fine tuned so as to overcome the disadvantages existed in the prior art with an artful combination means. The combination of the high resolution screen of the smartphone further provides an infinite possibility vision table or code display used for the visual acuity test. Moreover, the combination of the smartphone is able to assist the real time analysis of the test result and the real time communication of the test result and also allows the test result to be stored in an electronic device.

The first prior art also provides a solution of self managing visual acuity test, in which the generated result is similar to a visual acuity test which is an existed technology used in an office of a doctor. Through the published binocular viewer works with the smartphone for processing a certain application program, the user can perform a long-distance (or short-distance) visual acuity test for self management without any additional assistance. Moreover, the system assembled by the binocular viewer and the smartphone can also be used in other visual test including contract sensitivity, color sensitivity and refraction error.

The first prior art discloses a system presenting visual patterns via an optical system, which includes: a) a housing; b) a pair of lens tubes disposed in the housing; c) each second lens in visual communication with the lens tube separately; d) each first lens in visual communication with the second lens separately and the first lens including a front and a back lens surfaces; e) a front cover allowing a screen of an electronic equipment to be disposed, and the screen of the electronic equipment is an optical plane of the front lens surface of the first lens; f) comparing the patterns formed by the lens in the first lens tube and the lens in the second lens tube for presenting to the pattern in the optical system so as to generate the visual differences in the horizontal angle.

The technology feature of the first prior art is different from that of the present invention. The aforesaid invention can only be used to perform the visual test to confirm whether the visual response effect of the eyes of the patient being normal, and it is unable to obtain the eye prescription via an optometry means; thus, the patient is not provided with the optometry data required by the patient who wants to customize a pair of glasses.

The second prior art has disclosed a myopia prevention item used for preventing the axial myopia from being caused and delaying the deterioration of the axial myopia. The myopia prevent items include a light-transmitting part; characterized in that: the light-transmitting part allows light with the wavelength of 350 nm˜400 nm to penetrate, the light-transmitting part allows the light with the wavelength of 350 nm˜400 nm having a light intensity below 5.0 mW/cm² to penetrate. Wherein, the light-transmitting part does not allow the light having the wavelength below 315 nm to penetrate. Wherein, the light-transmitting part allows the light having the wavelength exceeding 400 nm to penetrate.

The second prior art also discloses a myopia prevent item used for preventing the

axial myopia from being caused and delaying the deterioration of the axial myopia, and includes a light-emitting part; characterized in that: light-emitting part at least emits light having the wavelength of 350 nm˜400 nm. Wherein, the light-emitting part at least emits light having a peak with an intensity variable range within a range of 350 nm˜400 nm. Wherein, the light-emitting part does not emit light with the wavelength smaller than 350 nm.

Wherein, the light-emitting part emits the light having an intensity lower than 5.0 nW/cm². The extension eye prescription of the eye axial length of the eye that receives ultraviolet rays exceeding 315 nm and below 400 nm among the ultraviolet rays is obviously smaller compared to the extension eye prescription of the eye axial length of the eye that does not receive ultraviolet rays of all wavelengths, and myopia eye prescription of the refraction of the eye is smaller. In other words, the myopia can be prevented through allowing the eye being exposed to the ultraviolet rays composed by certain wavelengths and bearable by the eye, so that effects of preventing the myopia from being caused and delaying the deterioration of the myopia are provided, and the aforesaid situation is firstly discovered. As such, an objective of the present invention is to provide an axial myopia prevention item, and the axial myopia prevention item allows to receive ultraviolet rays exceeding a wavelength of 315 nm and below 400 nm or ultraviolet rays a wavelength of above 360 nm and below 400 nm.

One embodiment of the second prior art is to disclose an article for myopia prevention (except Artiflex (trade name)), which includes: a light penetrating part that allows ultraviolet rays with a wavelength of exceeding 315 nm and below 400 nm to penetrate, and allows ultraviolet rays with a wavelength of below 315 nm cannot penetrate. A myopia prevention article is also provided and includes: a light penetrating part that allows ultraviolet rays with a wavelength above 360 nm and below 400 nm to penetrate, and prevents ultraviolet rays with a wavelength below 360 nm from penetrating.

The technology feature of the second prior art is different from that of the present invention. The second prior invention does not disclose a method of how to actually prevent the ultraviolet rays from penetrating; meanwhile, the second prior invention only discloses the means of preventing myopia by blocking ultraviolet rays within a certain range of wavelength, which is only one of the means for the purpose of preventing myopia. The present invention utilizes a means of continuously monitoring the data of the eye axial length; thus, an advantage of preventing the myopia from being caused and saving the treatment cost is provided. Meanwhile, the present invention works with the matched doctor and the optometry institution to directly obtain data, and the cooperating firms or institutions having the qualification for customizing glasses can directly allow the patient to customize the peripheral defocus glasses (such as an orthokeratology lens or glasses) to control the myopia eye prescription.

The third prior art has disclosed a method and a device for treating a human child having myopia. For example, a human child having myopia can be treated by wearing spectacles having one or two myopia control spectacle lenses. By wearing the spectacles having myopia control spectacle lenses to treat the child having or possibly having the myopia problem, a safe, effective and non-invasive method is provided for decreasing the myopia progression of the child and underaged people.

The third prior art also discloses a spectacle for reducing the progression of myopia in human children, the spectacles comprising at least one myopia control spectacle lens, the at least one myopia control spectacle lens comprising a monovision zone and a myopia control zone, wherein the myopia control spectacle lens comprises (i) a pass optical center, and (ii) the surrounding annular portion, both (i) and (ii) are substantially centered on the lens axis, the clear aperture corresponds to the single vision area and the annular area corresponds to the myopia control area and includes one or more features that reduce the visual acuity of the corresponding eye; and wherein the one or more features include scattering centers, and the scattering centers may include random variations in the spacing between adjacent dots and/or random variation in dot size change the pattern to configure. The third prior art also discloses a spectacle for reducing the progression of myopia in human children, the spectacles comprising at least one myopia control spectacle lens, the at least one myopia control spectacle lens comprising a monovision zone and a myopia control zone, wherein the myopia control spectacle lens comprises (i) a pass optical center, and (ii) the surrounding annular portion, both (i) and (ii) are substantially centered on the lens axis, the clear aperture corresponds to the single vision area and the annular area corresponds to the myopia control area and includes one or more features that reduce the visual acuity of the corresponding eye, and wherein the central region has a diameter ranging from 2 mm to 8 mm.

The third prior art also discloses a spectacle for reducing the progression of myopia in human children, the spectacles comprising at least one myopia control spectacle lens, the at least one myopia control spectacle lens comprising a monovision zone and a myopia control zone, wherein the myopia control spectacle lens comprises (i) a pass optical center, and (ii) the surrounding annular portion, both (i) and (ii) are substantially centered on the lens axis, the clear aperture corresponds to the single vision area and the annular area corresponds to the myopia control area and includes, one or more features corresponding to visual acuity of the eye are reduced, and wherein the central region is aligned with the front of the human pupil when staring at a distance.

The technology feature of the third prior art is different from that of the present invention. The third prior invention utilizes a defocus design to achieve an effect of preventing the eye axial from being prolonged, but a calibrating operation is still required to be performed to a glasses frame. As such, the third prior invention may be limited to different glasses frames selected by the user, and a limitation is generated. The present invention works with the matched doctor and the optometry institution to directly obtain data, and the cooperating firms or institutions having the qualification for customizing glasses can directly allow the patient to customize the peripheral defocus glasses. Meanwhile, a follow-up consulting matching service is provided, so that the professional doctor is introduced to the patient, and the effective application for the professional sources is established.

The fourth prior art has disclosed a process and an apparatus for determining optical aberrations of an eye; the eye has an optical system including the cornea and the lens. The process includes the reconstructing of wavefront aberrations of the eye as a deviation of the wavefront, determined by the optical system of the eye with a process of aberrometry, with respect to an ideal planar wavefront generated by an aberration-free eye model. A measured ocular length is employed for the aberration-free eye model.

The fourth prior invention also discloses a method used for the optical aberration for determining eyes, including:

The length of the eyes is measured to produce the eye length (198, OL) of measurement; the aberrationless eye model with model lens, model retina and model eye length is built in the following manner: selecting from a distance of the summit on the rear surface of model lens to model retina so that model eye length corresponds to measured eye length (198, OL); the wavefront aberration of the eyes is defined as the optical system of the eyes using an aberration measurement deviation of the wave surface that the wave surface caused by is generated with respect to the aberrationless eye model; the model is the eye length (198, OL) of the measurement as the model eye length.

An embodiment of the fourth prior art also can be specifically concluding aberration up to fourth order Zenike (cylindricality aberration, broom difference, shamrock pattern aberration, cloverleaf pattern aberration, and spherical aberration) reaching more preferable near vision and to be applied to correction presbyopia. The method includes following steps: (a) generating the multiple single beam component of directional light shafts, and the beam component has two-dimensional arrangements; (b) utilizing the optical system of the eyes to the beam component is projected so as in the eyes, the first dot pattern of the beam component projection is generated on retina; (c) first dot pattern is projected to the second plane being arranged on an outside of the eyes by an ophthalmoscope device so as to allow the second projection of the first projection by the beam component generated in step (b) generates the second dot pattern; (d) recording the second pattern on second plane; (e) building an aberrationless eye model with a model eye length by calculating, the model eye length is numerically using the actual eye length of measurement; (f) each second projection of the beam component being recorded in the second plane, in second dot pattern, the beam component ophthalmoscope projection generate with respect to the eye model of construction, same point of the two-dimensional arrangements, and the skew of the projection of amount light beam is measured; and (g) wavefront aberration of the optical system of eyes is rebuild as the wave surface phase by being generated by the optical system, the deviation of the wave surface generated by aberrationless eye model.

The technology feature of the fourth prior art is different from that of the present invention. The fourth prior invention only discloses a calibration obtaining method for the data of the measured eye axial length. The present invention is able to process the eye axial length measure which is integrated with services provided by the optometry institution or glasses shop to allow the patient to directly finish his/her needs of customizing a pair of glasses. The fourth prior invention is one of the applicable methods for calibrating an instrument comparing to the present prior art.

The fifth prior art has disclosed a blink detection algorithm for the ophthalmic lens including electronic system and associated circuit. The blink detection algorithm realizes that the system controller is bonded to a part for the electronic system in the ophthalmic lens in the system controller. The electronic system includes one or more batteries or plural power suppliers, electric power management circuit, one or more sensors, a clock generation circuit, a control algorithm and circuit and a lens drive circuit. The blink detection algorithm realizes following steps: with set rate to the light sampling being incident in individual eyes and the sample at least temporarily preserving collection, determine when eyelid is opened or closed to predict number of times, time cycle and the pulse width of the blink from the sample collected, the duration of the number of times and blink blinked in cycle preset time is calculated, and the time in the number of times blinked in cycle preset time, the duration of blink and cycle preset time suddenly is compared to determine blink pattern with the sample group of storage.

The fifth prior art also discloses a method of detecting blinked eye and blink pattern, the method includes: with a set rate to the light sampling being incident in individual eyes and the sample at least temporarily preserving collection; determine when eyelid is opened or closed to predict number of times, the time cycle of the blink of the sample from the collection and pulse width; calculate the duration of the number of times and blink blinked in cycle preset time; by in number of times, the duration of the blink and cycle preset time blinked in cycle preset time suddenly compares to determine blink with the sample group for representing one or more storage of predetermined intentional blink sequence pattern; and determine whether the blink corresponds to one or more in the predetermined intentional blink sequence, and characterized in that, “determining whether the blink corresponds to one or more in the predetermined intentional blink sequence” step includes sequentially allowing the deviation to the blink from the predetermined intentional blink sequence by implementation blink “sheltering”.

The fifth prior art further discloses a method for detecting the system blinked and blink pattern, the system includes: a photodetector, the photodetector is configured to the signal that output corresponds to the intensity of the light being incident on eyes; an amplifier, the amplifier is configured to receive the signal from the photodetector and is configured to improve its power electricity it is flat that the amplification signal for corresponding to the light being incident on the eyes is produced with further treatment; and a processor, the processor is configured to receive the amplified signal, and the processor is sampled with set rate, and at least the sample collected temporarily is preserved, determines when eyelid is opened or closed to predict the blink of the sample from the collection number of times, time cycle and pulse width, calculate the duration of the number of times and blink blinked in cycle preset time, will it is blinked in number of times, the duration of the blink and cycle preset time blinked in cycle preset time between time compare to determine blink pattern with the sample group of storage, and determine whether the blink corresponds to one or more intentional blink sequence, and characterized in that, “determining whether the blink corresponds to one or more blink sequence intentionally” is included by implementing to blink “sheltering” sequentially allows the deviation of the blink to sequence of being blinked intentionally from described one or more.

The technology feature of the fifth prior art is different from that of the present invention. The fifth prior invention provides a blink detecting system.

Based on what has been disclosed above, how to provide a monitoring and activity matching system for eye health, in which the underaged person is able to finish the measure for obtaining the prescription changes within a short period of time (about 20˜30 second) and no dilating test is required, so that the parents can easily monitor the eye health of the underaged person, and to match an ophthalmologist or a nearby optometry institutions to allow the patient to directly finish the glasses customization, and to effectively applies the spared sources of the existed optometry institutions and the glasses shops shall be improved by the skilled people in the arts.

### SUMMARY OF THE INVENTION

For solving the existed problems and disadvantages in the conventional technology the present invention provides a monitoring and activity matching system for eye health, which is capable of obtaining the data of an eye axial length of a patient with a non-contact means; meanwhile, spared sources of the existed optometry institutions and the glasses shops can be efficiently applied.

Another objective of the present invention is to provide a monitoring and activity matching system for eye health, in which an eye axial length detecting module has a converting database of the measured eye axial length established by corresponding to the eye developing progression along with the ages of the children, the data of the eye axial length obtained through detecting and measuring the eye axial length through the instrument of the eye axial length detecting module analyzes different eye axial length values, and the values are converted according to the predicted corresponding myopia dioptors obtained after cycloplegic method so as to establish a converting standard which is able to compare the changes of the eye axial length values with the date in the database of converted measured eye axial length; meanwhile, the accuracy of the database of converted measured eye axial length corresponding to the myopia dioptors can be increased through the data being continuously accumulated and modified.

For achieving the aforesaid objective, the monitoring and activity matching system for eye health of the present invention includes: an eye axial length detecting module used for detecting the data of an eye axial length of a patient; a member system used for inputting the data of the eye axial length; and a follow-up consulting and activity matching system used for allowing the patient to be matched with a follow-up consulting and an activity according to the data of the eye axial length.

For achieving the aforesaid objective, the eye axial length detecting module of the monitoring and activity matching system for eye health of the present invention utilizes an eye axial length detecting technology to obtain the data of the eye axial length of the patient with a non-contact manner.

For achieving the aforesaid objective, the eye axial length detecting module of the monitoring and activity matching system for eye health of the present invention includes at least one eye axial length detecting instrument, and the at least one eye axial length detecting instrument includes, but not limits to, a non-contact type optical eye axial length measuring instrument.

For achieving the aforesaid objective, the member system of the monitoring and activity matching system for eye health of the present invention is a data and record center in which the eye axial length, the follow-up consulting and the activity of the patients including, but not limiting to, the data of the eye axial length, the data of follow-up consulting and activity.

For achieving the aforesaid objective, the follow-up consulting and activity matching system of the monitoring and activity matching system for eye health of the present invention is used to integrate including, but not limiting to, the matching of the health activity and the matching of the hospital or clinics for follow-up consulting.

For achieving the aforesaid objective, the follow-up consulting and activity matching system of the monitoring and activity matching system for eye health of the present invention is used for providing operations of booking the matched follow-up consulting or activity and the record of finished activity, or providing the patient with the advanced service requirement for eye health of the patient.

For achieving the aforesaid objective, the follow-up consulting and activity matching system of the monitoring and activity matching system for eye health of the present invention is used for matching an activity including, but not limiting to, customizing a pair of glasses or contact lenses and an operating firm and matching a person including, but not limiting to, a doctor, an optometrist, a patient who needs an optometry test, a firm, an optometry institution or a glasses shop and a health resource.

For achieving the aforesaid objective, the member system and the follow-up consulting and activity matching system of the monitoring and activity matching system for eye health of the present invention is able to perform a comprehensive health monitoring and a practical application through an application program.

For achieving the aforesaid objective, the application program of the monitoring and activity matching system for eye health of the present invention includes an eye health bank concept application, an eye health online service integration, an eye axial length measuring instrument and service calibrating mechanism, a footprint grading system, and an eye health medical big data establishment.

For achieving the aforesaid objective, the member system of the monitoring and activity matching system for eye health of the present invention is able to convert values including, but not limiting to, the data of the measured eye axial length, the record of the eye axial length changes, the data of follow-up consulting and activity into a healthy behavior award point applicable to an eye health bank application, and an eye health bank concept mechanism is established through the healthy behavior award points.

For achieving the aforesaid objective, the eye health online service integration of the monitoring and activity matching system for eye health of the present invention includes eye health services such as measuring eye axial length, optometry checkup and customizing a pair of glasses or contact lenses.

For achieving the aforesaid objective, the eye axial length measuring instrument and service calibrating mechanism of the monitoring and activity matching system for eye health of the present invention is able to use an objective service review to optimize professional service skills and staff attitudes, and the contains in the objective service review is used to establish educational courses and make an calibration education at a fixed timing for an eye axial length measuring instrument.

For achieving the aforesaid objective, the application program of the monitoring and activity matching system for eye health of the present invention is used to obtain an activity footprint of a patient for establishing a publicity for activity sharing and review.

For achieving the aforesaid objective, the eye health medical big data establishment of the monitoring and activity matching system for eye health of the present invention is able to use the follow-up consulting and activity matching system to integrate data including, but not limiting to, the data of the measured eye axial length, the record of the eye axial length changes, the data of follow-up consulting and activity of all patients to establish a model of patient changes and consuming footprints so as to establish an eye health medical big data for the whole human beings.

### BRIEF DESCRIPTION OF THE APPTERMINALED DRAWINGS

FIG. 1 is a flowchart showing the monitoring and activity matching system for eye health according to the present invention.

FIG. 2 is an embodiment in which a model being structure by an automated program with the data of the eye axial length.

### BRIEF DESCRIPTION OF THE DRAWINGS

100: Monitoring and activity matching system for eye health; 200: Eye axial length detecting module; 300: Member system; 400: Follow-up consulting and activity matching system.

### DETAILED DESCRIPTION OF THE INVENTION

The technical contents of this disclosure will become apparent with the detailed description of embodiments accompanied with the illustration of related drawings as follows. It is intended that the embodiments and drawings disclosed herein are to be considered illustrative rather than restrictive.

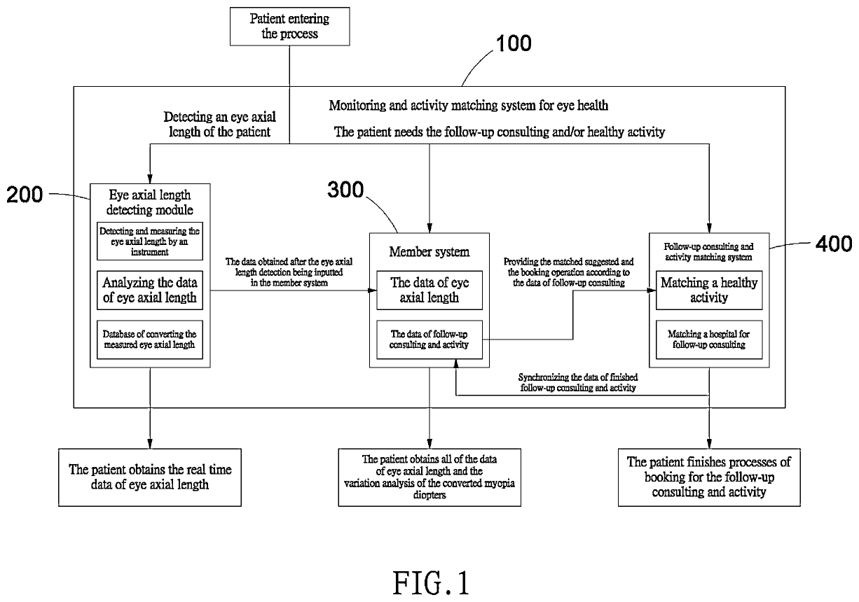

Please refer to FIG. 1, the present invention provides a monitoring and activity matching system for eye health 100, which includes an eye axial length detecting module 200, a member system 300 and a follow-up consulting and activity matching system 400. Wherein, the eye axial length detecting module 200 is used for detecting the data of the eye axial length of a patient, the member system 300 is used for inputting the data of the eye axial length, and the follow-up consulting and activity matching system 400 is used for making a follow-up consulting and activity matching process according to the data of the eye axial length of the patient.

Accordingly, the monitoring and activity matching system for eye health 100 provided by the present invention is able integrate the obtained current healthy condition according to the eyes of the patient, the record of the eye axial length changes and the follow-up consulting and activity service for establishing an integrated system. The monitoring and activity matching system for eye health 100 includes, but not limits to, the eye axial length detecting module 200, the member system 300 and the follow-up consulting and activity matching system 400. After the data of the real-time eye axial length of the patient is detected by the eye axial length detecting module 200, the data is inputted in the member system 300, thus the patient can obtain the record of the eye axial length changes and the evaluation of the myopia refraction changes. When the patient himself/herself desires to have a follow-up consulting or maintaining the condition of eyes or a body health activity, or an abnormal situation found in the follow-up consulting data of the patient from the member system 300 of the monitoring and activity matching system 100 (for example, the change of the myopia refraction is too much and an effect of establishing digital doctor referral), the follow-up consulting and activity matching system 400 can be actuated or triggered to perform matching the suitable healthy activity or making a follow-up consulting to a medical institution.

Details are provided as follows. The eye axial length detecting module 200 of the monitoring and activity matching system for eye health 100 provided by the present invention utilizes an eye axial length detecting technology (similar to an instrument technology of optically measuring the eye axial length with a computerized measuring manner) which is a non-contact manner to obtain the data of the eye axial length of the patient (including, but not limiting to, children and adult). The eye axial length detecting module 200 includes at least one eye axial length detecting instrument for detecting and measuring the eye axial length, and the at least one eye axial length detecting instrument includes, but not limits to, a non-contact type optical eye axial length measuring instrument used for achieving the eye axial length detecting operation.

According to the present invention, the eye axial length detecting module of the monitoring and activity matching system for eye health has a database of converting the measured eye axial length, which is established with respect to the normal eye health levels acquired corresponding to the eye developing process along with the ages of the children, the data of the eye axial length obtained through detecting and measuring the eye axial length through the instrument of the eye axial length detecting module analyzes different eye biometric values, and the values are converted according to the predicted corresponding myopia diopters obtained after cycloplegic method so as to establish a converting standard which is able to compare the changes of the eye biometric values with the date in the database of converted measured eye axial length; meanwhile the accuracy of the database of converted measured eye axial length corresponding to the myopia refraction can be increased through the data being continuously accumulated and modified.

The converting standard can utilizes a multiple regression analysis to achieve the changes of the eye biometric values; the multiple regression analysis adopts two or more independent variables and only one dependent variable Y to calculate and analyze a relation of multiple (two or more) independent variables and a dependent variable Y; the refraction error is defined as the dependent variable Y to simulate and search for the most suitable predictable variable X, and a model of the parameter of the predictable variable being structured is illustrated with the following embodiment:

The least square difference is used for structuring the model [symbol] = [symbol] ( [symbol] − [symbol] )², wherein
[symbol] is the model predicted value.
[symbol] is the correct training data.

Taking [symbol] as the minimum value for the linear line for structuring the regression model.

As such, a predicating linear model closest to the data is generated, the linear model taking the minimum value is the predicating line closest to the data; steps for structuring the model is disclosed by the embodiment, as follows;
1. Providing the parameters (predicable variables) of the refraction errors which are defined as predicable in a model, a residual least square manner is adopted to structure the model.
2. The residual in the model is processed with a basic model test such as a normality test, and then a stepwise regression means is processed for variable selection; with the variable explanatory and the variable quantity control, variables such as Age, AL (defining as a front and rear radial length of eye axle), K1 (defining as a horizontal curvature of the cornea), K2 (defining as a vertical curvature of the cornea), ACD (defining as an anterior chamber measuring depth), LT (defining as a lens thickness) are adopted for structuring the model.
3. Utilizing the variable inflation factor (VIF) to determine whether the model has the collinearity, so that the collinearity is avoided from affecting the explanatory and prediction of the explanatory variables in the model.

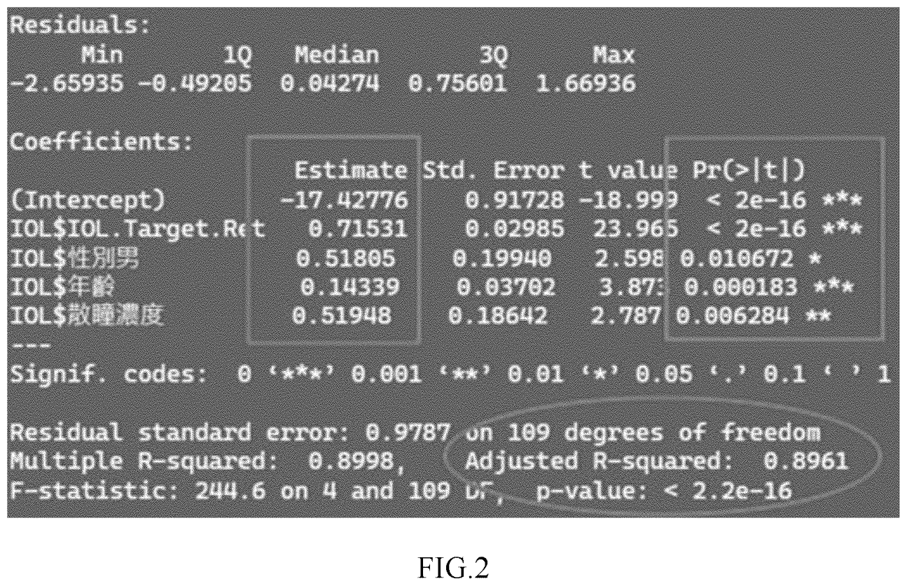

Please refer to FIG. 2, the followings are the embodiment in which a model being structure by an automated program with the data of the eye axial length:

The data used in the regression model is the eye axial length of the patient obtained after the pupils of the patient are sufficiently dilated to eliminate excessive accommodation the real refraction error, the age, the anterior chamber measuring depth, the lens thickness and the cornea horizontal value K which are adopted to process a model training prediction; thus the regression model structured: is the refraction errors predicted=102.559+(−2.7982)*AL+0.1219*Age+(−0.5879)*K1+(−0.2723)*K2+2.7685*ACD+(−3.4206)*LT+εε, wherein εε is the random error value of the model.

The slope of the variable “AL” is −2.7982, which means that the eye refractions will decrease 2.7982 whenever one unit being increased.

The slope of the variable “ACD” is 2.7685, which means that the eye refractions will increase 2.7865 whenever one unit being increased.

The model can explain 91.7% (Adjusted R-squared: 0.9167) of the refraction errors variation, which means that the four variables contained in this model can explain about 92% of the refraction errors variation.

According to the present invention, the member system 300 of the monitoring and activity matching system for eye health 100 is a data and record center in which the eye axial length, the follow-up consulting and the activity of the patients (including the patients participating the present invention or other obtainable data source) includes, but not limits to, the data of the eye axial length, the data of follow-up consulting and activity. Moreover, the member system 300 provides the patient with the record of the eye axial length changes of the patient himself/herself and the evaluated changes of the myopia refraction, and the historical activity and the follow-up consulting data and records. The member system 300 is even able to establish full-age eye monitoring, big data analysis for time periods according to the data of the eye axial length of the patient, so that the whole matching or requiring of the data and the follow-up consulting and activity of the patient can be established.

According to the present invention, the follow-up consulting and activity matching system 400 of the monitoring and activity matching system for eye health 100 can integrate including, but not limiting to, the matching of the health activity and the matching of the hospital for follow-up consulting, providing operations of booking the matched follow-up consulting or activity and the record of finished activity, or providing the patient with the advanced service requirement for eye health, thus the matching for medical institution and the matching for activity are provided.

The activity included in the follow-up consulting and activity matching system 400 includes, but not limits to, customizing and/or adjusting glasses or contact lenses (the optometrist performs a fine prescription adjustment and a both eye balancing test), and the operating firm and the matched personnel/firm includes, but not limits to, a doctor (an ophthalmologist or an optometrist), the patient who needs an optometry test, a firm, an optometry institution or a glasses shop and a health resource (foods, sports, advertisements).

According to the present invention, the member system 300 and the follow-up consulting and activity matching system 400 of the monitoring and activity matching system for eye health 100 can perform a full health monitoring and the practical application of a patient through an application program (APP). Based what has been disclosed in the embodiment, technical characteristics and effects are provided as follows:

First technical feature: The patient can access real-time data on the axial length of the eye through the eye axial length detection module 200. This data can be obtained after completing examinations at a medical facility or undergoing a mydriatic process conducted by a professional doctor to check for myopia diopters. Subsequently, an application program (APP) can be utilized to create an account based on the initial eye axial length data or the initial data of tested myopia diopters post mydriatic process. This data can then be used to establish an eye health bank mechanism, enabling the patient to maintain a personalized eye health record.

Second technical characteristic: the patient can go to a firm or skilled institution cooperating with the present invention including, but not limiting to, an optometry institution, a glasses shop having the eye axial length detecting instrument to periodically measure the eye axial length. The data obtained after the measurement will also be uploaded to the member system 300 of the present invention, and the patient can browse the record of the eye axial length changes at any desired time via the application program (APP).

Third technical feature: The operating hours and off-peak periods of the collaborating establishments, including optometry institutions and glasses shops, can be communicated to the patient through the application program (APP). For instance, the patient may receive notifications containing information about the optometry institution or glasses shop they visited for follow-up consultations, as well as details about off-peak times. This enables the patient to schedule periodic eye axial length measurements at the institution or shop, or use the application program (APP) to locate nearby facilities offering eye axial length detection services and book appointments through the positioning system. Consequently, the patient can easily identify a collaborating institution for long-term or specific duration eye axial length monitoring, facilitating continuous monitoring of the patient's eye axial length.

Fourth technical characteristic: the member system 300 is able to convert data including, but not limiting to, the data of the measured eye axial length, the record of the eye axial length changes into an indication through the application program (APP) (for example, defined as an eye developing indication and presenting with an award point manner).

Fifth technical characteristic: the follow-up consulting and activity matching system 400 is able to convert the data of the eye changes of the patient himself/herself into an indication, and the firm or the skilled institution including, but not limiting to, the optometry institution, the glasses shop having the eye axial length detecting instrument are able to use the eye axial length detecting module 200 to perform an eye axial length measurement so as to process the health activity (for example, a mode of getting a discount via the accumulated award points so as to prompt the products in the participated optometry institution or glasses shop).

Sixth technical characteristic: When the change of the eye axial length in the member system 300 is obvious (defined by axial length elongation per year according to patient's age), the application program (APP) is able to send a notification to the patient to remind the patient going to the cooperating medical institution through the follow-up consulting and activity matching system 400 to book for a follow-up consulting or a referral; meanwhile, the record of the eye axial length changes is synchronously provided to the medical institution as a treatment reference.

Seventh technical characteristic: the application program (APP) is able to integrate all the experience reviews provided by all the patients who go there for the periodical eye axial length measurement through the eye axial length detecting module 200 including, but not limiting to, the optometry institution, the glasses shop having the eye axial length detecting instrument, and the experience review provided by the patient who had the follow-up consulting and participated the activity though the follow-up consulting and activity matching system 400, so that better determining benchmarks for the operating firms and institutions can be provided (for example, when the patient needs to customize a pair of normal or special glasses such as defocus glasses at his/her own area, the present invention is able to provide the experience reviews to the optometrist/ophthalmologist performing the periodically follow-up consulting as a reference, referral the patient to the suggested activity or the cooperating firms or institutions to meet the requirements of the patient for customizing a pair of normal glasses or a special glasses such as the defocus glasses to control the myopia).

Eighth technical characteristic: the follow-up consulting and activity matching system 400 is able to use the application program (APP) to integrate including, but not limiting to, a healthy lifestyle advertisement, a nutrition health product promoting activity, a sport group activity promotion to provide sources of eye health activities.

The application program of the monitoring and activity matching system for eye health 100 provided by the present invention further includes an eye health bank application, an eye health online service integration, an eye axial length measuring instrument and service calibrating mechanism, a footprint grading system, and an eye health medical big data establishment; the details are provided as follows:

First, an embodiment illustrating the benefit method of the eye health bank application of the present invention and the matching activity:

The member system 300 of the monitoring and activity matching system for eye health 100 provided by the present invention is able to convert values including, but not limiting to, the data of the measured eye axial length, the record of the eye axial length changes, the data of follow-up consulting and activity into a healthy behavior award point applicable to the eye health bank application (for example, the health award point corresponding to the eye axial length changes being greater or smaller is presented as a health deposit amount like an actual online bank, but the award point is defined as healthy behavior award points of the patient), and a health bank mechanism is established through the healthy behavior award points (the patient has his/her own health bank book). The healthy behavior award points of the patient can be synchronized with family members or friends or a certain person to establish a mutually tracking the health award point mechanism (for example, the healthy behavior award points of children can be synchronized with the bank book of the parents to allow the parents to see and assist the tracking of the visual condition of the children). The patient can be provided with additional services or a discount for including, but not limiting to, the follow-up consulting, the activity and the eye axial length measurement through the accumulated award points, so that the patient will have the participating motivation for eye health activity; meanwhile, the cooperating firms or institutions who provides the activity will have chances to make the advertisement be seen to prompt the products or services, thus the present invention is able to get benefits (for example, revenue share) through the cooperating firms or institutions having the advertisement being seen to prompt the products or services.

Second, an embodiment illustrating the eye health online service integration for measuring eye axial length, optometry checkup and customizing glasses:

The eye health online service integration of the monitoring and activity matching system for eye health 100 provided by the present invention includes eye health services such as measuring eye axial length, optometry checkup and customizing glasses. As such, the follow-up consulting and activity matching system 400 is able to integrate the services of follow-up consulting and the activity provided by the cooperating firms or institutions. When the patient wants to customize a pair of glasses, the system is able to provide the required glasses customizing information to the referral optometrist/ophthalmologist, or a local firm may be recommended, and the patient can freely choose including an optometry institution or a glasses shop (for example, being suitable, close to home or not, whether having the corresponding glasses and/or frame, and being capable of customizing the special defocus optical glasses or not) to allow the patient to freely select so as to finish the glasses customization. Moreover, this embodiment can be integrated with the aforesaid “First, an embodiment illustrating the benefit method of the eye health bank application of the present invention and the matched activity”, so that the patient can be provided with the accumulated award points so as to have additional services or discount which includes, but not limits to, customizing glasses.

Third, an embodiment illustrating the eye axial length measuring instrument and service calibrating mechanism:

The eye axial length measuring instrument and service calibrating mechanism of the monitoring and activity matching system for eye health 100 provided by the present invention is able to use an objective service review to optimize the service skills and service attitude, and the contains in the objective service review can be used to establish education classes and make a calibration education at a fixed timing for the eye axial length measuring instrument.

Details are provided as follows. The objective service reviews for the cooperating firms, the optometry personnel, the optometry institution and the glasses shop can be established through the experience reviews stored in the application program (APP) to optimizing the professional service skills and service attitude. Meanwhile, the contains in the reviews can be used to establish education classes and make a calibration education at a fixed timing for the eye axial length measuring instrument (the aforesaid classes, education can be presented with means including, but not limiting to, an online education video, audio, and illustrations with pictures and texts), so that the monitoring stability and uniformity can be maintained. The mechanism may also allow the firms to be provided with a capability of calibrating the instrument at a fixed timing with at a highest standard through lending or leasing the instruments including, but not limiting to, the eye axial length instrument and the visual instrument and the aforesaid education classes, the calibrating education being provided to the firms to finish the educational training.

Fourth, the footprint grading system:

The activity footprint of the patient obtained via the application program (APP) of the monitoring and activity matching system for eye health 100 provided by the present invention is able to be used to establish the publicity for activity sharing and reviews.

Details are provided as follows. The discount for follow-up consulting or activity can be directly obtained through the present invention (for example, the patient gets the discount through scanning the specified QR code), and the patient provides the service review; thus, the publicity for activity sharing and reviews can be established through all the activity footprints of the patient participating the present invention. The present invention can also work the review publicity (for example, social media sharing), and a revenue sharing mechanism (for example sharing any of the aforesaid “healthy behavior award point”) is created to increase the benefit sources for the cooperating firms, institutions, patient and the present invention.

Fifth, an embodiment illustrating the eye health medical big data establishment:

The eye health medical big data establishment of the monitoring and activity matching system for eye health 100 provided by the present invention can use the follow-up consulting and activity matching system to integrate data including, but not limiting to, the data of the measured eye axial length, the record of the eye axial length changes, the data of follow-up consulting and activity of all patients to establish a model of patient changes and consuming footprints so as to establish an eye health medical big data for the whole human beings.

Based on what has been disclosed above, the conventional myopia is hard to be determined at an early stage and unable to be tracked in the long term by utilizing the conventional technology, the reason is that when the patient has the visual acuity test due to his/her vision problem, especially for the underaged patient, the patient needs to go to the optometrist/ophthalmologist in a specialized clinics or hospital for a double check, however the amount of the eye specialized clinics/hospital is very few; take the data of students in the first grade in Taiwan as an example, there are 20 to 25% of the underaged people already have the myopia, and the percentages boosts up to 80 to 85% for the students in the twelfth grade, thus the detecting capacity of the existed amount of the eye specialized clinics/hospitals is not enough. Moreover, there is a problem that the underaged people may have a early-stage myopia, and the mydriatic eyedrops has to be used to decrease the excess accommodation to process the precise optometry checkup so as to determine to myopia diopters, the required time is very long (needing to wait about one hour and then the optometry checkup can be processed), and the patient may feel tingling after the mydriatic eyedrops is applied in the eyes, so that the underaged people may not want to have the continuing the long time tracking.

As such, the data of the eye axial length of the patient can be measured with a non-contact means through the eye axial length detecting module 200 of the present invention with the indirect eye axial length measuring instrument (an optical optometry instrument) performing the measurement so as to obtain the myopia diopter changes of the patient. Accordingly, the underaged people can finish the measurement for obtaining the diopter changes within a short period of time (about 20˜30 second) and no mydriatic eyedrop is required.

On the other hand, take Taiwan as an example, there are about 5,000, including the optometry institutions and the glasses shops distributed in various cities and counties of Taiwan. The optometry institutions and the glasses shops mainly focus on selling products such as glasses frames, and not providing with the eye health service. As such, the optometry institutions and the glasses shops will often have the off peak time periods which cause the unnecessary operation costs, and the optician personnel may not be able to provide the sufficient eye health service with his/her professions to confirm the current status of the patient and the health care actions possibly be required in the future. Accordingly, the opticians in optometry institutions or the glasses shops want to work with the optometrist/ophthalmologist, but there is no systematic solution to solve the problem at the current stage.

However, with the follow-up consulting and activity matching system 400 provided by the present invention, a modelized manner is used to connect the cooperating firms, institutions and the optometrist/ophthalmologist, and the member system 300 is able to provide the data including, but not limiting to, the data of the measured eye axial length, the record of the eye axial length changes, the data of follow-up consulting and activity of the patient, so that the optometrist/ophthalmologist can give the patient a proper prescription; finish the glasses customization finish the glasses or contact lenses customization and a suitable firm can be matched to allow the patient to finish the glasses customization, thus the spared sources of the existed optometry institutions and the glasses shops can be efficiently applied.

The converting database of the measured eye axial length of the eye axial length detecting module 200 of the present invention can effectively provide an integration of the normal eye test and the data of the eye axial length so as to provide a determine method for daily eye health maintenance; meanwhile, through the converting database of the measured eye axial length, a standard of the eye axial length changes can be converted into a predicted refraction diopters, and the application program (APP) of the present invention can practice the embodiment to establish a method illustration for establishing an eye health monitoring group, and the full records of the eye axial length changes of the patient can be continuously increase collecting the record data of the application program (APP) to realize a more precise converting standard of the axial eye length changes corresponding to the refraction diopters, and the comprehensive data of the eye axial length changes of more patient can be integrated through the eye axial length measuring instrument and service calibrating mechanism and the embodiment and the optometry institution having the eye axial length detecting instrument; as such, the precise converting standard of the eye axial length can be realized and achieved; in other words, the follow-up and activity matching system can provide a more precise activity suggestions (for example, reminding the patient spending less time on electronic devices, following the doctor orders, using the mydriatic eyedrops, regularly use the controlling type glasses or contact lens to achieve a controlling effect, and providing a follow-up booking table).

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific examples of the embodiments disclosed and that modifications and other embodiments are intended to be included within the scope of the appended claims. Although specific terms are employed herein, they are used in a generic and descriptive sense only and not for purposes of limitation.

What is claimed is:

1. A monitoring and activity matching system for eye health, including:
an eye axial length detecting module, used for detecting data of an eye axial length of a patient;
a member system, used for inputting the data of the eye axial length; and
a follow-up consulting and activity matching system, used for allowing the patient to be matched with a follow-up consulting and an activity according to the data of the eye axial length.

2. The monitoring and activity matching system for eye health as claimed in claim 1, wherein the eye axial length detecting module utilizes an eye axial length detecting technology to obtain the data of the eye axial length of the patient with a non-contact manner.

3. The monitoring and activity matching system for eye health as claimed in claim 1, wherein the eye axial length detecting module includes at least one eye axial length detecting instrument, and the at least one eye axial length detecting instrument includes, but not limits to, a non-contact type optical eye axial length measuring instrument.

4. The monitoring and activity matching system for eye health as claimed in claim 1, wherein the member system is a data and record center in which the eye axial length, the follow-up consulting and the activity of the patients including, but not limiting to, the data of the eye axial length, the data of follow-up consulting and activity.

5. The monitoring and activity matching system for eye health as claimed in claim 1, wherein the follow-up consulting and activity matching system is used to integrate including, but not limiting to, the matching of the health activity and the matching of a hospital for follow-up consulting.

6. The monitoring and activity matching system for eye health as claimed in claim 5, wherein the follow-up consulting and activity matching system is used for providing operations of booking the matched follow-up consulting or activity and the record of finished activity, or providing the patient with an advanced service requirement for eye health of the patient.

7. The monitoring and activity matching system for eye health as claimed in claim 5, wherein the follow-up consulting and activity matching system is used for matching an activity including, but not limiting to, customizing a pair of glasses or contact lenses and an operating firm, and matching a person including, but not limiting to, a doctor, an optometrist, a patient who needs an optometry test, a firm, or a glasses shop and a health resource.

8. The monitoring and activity matching system for eye health as claimed in claim 1, the eye health monitoring and activity matching system includes a member system and a follow-up consulting and activity matching system capable of conducting a comprehensive health monitoring of a patient and implementing practical applications through a dedicated application program.

9. The monitoring and activity matching system for eye health as claimed in claim 8, the application program comprises features such as an eye health bank application, integration of online eye health services, an eye axial length measuring instrument with service calibration mechanism, a footprint grading system, and the establishment of an eye health medical big data repository.

10. The monitoring and activity matching system for eye health as claimed in claim 9, wherein the member system is able to convert values including, but not limiting to, the data of the measured eye axial length, the record of the eye axial length changes, the data of follow-up consulting and activity into a healthy behavior award point applicable to an eye health bank application, and an eye health bank mechanism is established through the healthy behavior award points.

11. The monitoring and activity matching system for eye health as claimed in claim 9 further specifies that the eye health online service integration encompasses services related to eye health, including the measurement of eye axial length, optometry services, and the customization of eyeglasses or contact lenses.

12. The eye health monitoring and activity matching system described in claim 9 incorporates an eye axial length measuring instrument and service calibrating mechanism that utilizes an objective service review to enhance professional service skills and attitudes. The information gathered from the objective service review is utilized to develop educational programs and conduct calibration sessions at scheduled intervals for the eye axial length measuring instrument.

13. The monitoring and activity matching system for eye health as claimed in claim 9, wherein the application program is used to obtain an activity footprint of a patient for establishing publicity for activity sharing and review.

14. The monitoring and activity matching system for eye health as claimed in claim 9, wherein the eye health medical big data establishment is able to use the follow-up consulting and activity matching system to integrate data including, but not limiting to, the data of the measured eye axial length, the record of the eye axial length changes, the data of follow-up consulting and activity of all patients to establish a model of patient changes and consuming footprints so as to establish an eye health medical bigdata for whole human beings.

15. The eye health monitoring and activity matching system described in claim 1, includes an eye axial length detecting module that incorporates a database for converting the measured eye axial length. This database is designed to align the measured eye axial length with established levels of normal eye health corresponding to the developmental progression of the eye in children of varying ages. The eye axial length data obtained through the detection and measurement process of the module is analyzed to identify different eye axial length values, which are then converted based on myopia dioptors obtained after cycloplegic method. This conversion process establishes a standard for comparison, enabling the tracking of changes in eye axial length values against the converted data stored in the database. Moreover, the accuracy of the database in relation to converted eye axial length measurements corresponding to myopia dioptors can be enhanced through continuous accumulation and refinement of data.
